(19) United States
(12) Patent Application Publication
(10) Pub. No.: US 2006/0264422 A1
(43) Pub. Date: Nov. 23, 2006

(54) Use of taurolidine as enhancer of the complement system

(76) Inventors: Johannes Reinmueller, Wiesbaden (DE); Daniel Rittirsch, Roemerberg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/429,437

(22) Filed: May 8, 2006

Related U.S. Application Data
(60) Provisional application No. 60/678,796, filed on May 9, 2005.

(51) Int. Cl. A61K 31/549

(52) U.S. Cl. 514/222.5

(57) ABSTRACT

Novel uses of taurolidine based on its ability to activate the complement system are described.

This application claims the benefit of the filing date of U.S. Provisional Application Ser. No. 60/678,796 filed May 9, 2005.

The chemotherapeutic agent taurolidine (4,4′-methylenebis-(1,2,4-thiadiazine-1,1-dioxide)) is known for its bactericidal, viricidal and fungicidal properties. Furthermore, it inhibits endotoxins, components of the blood coagulation system and individual components of the complement system. Modulation of the adhesion formation in the abdominal cavity after surgical interventions and the inhibition of the spreading of metastases in tumor disorders have also been described.

By virtue of the inhibiting effect of taurolidine on endotoxins of certain bacteria species, it was used in the past in medicine as an intravenous infusion for the therapy of septic shock.

More recent findings concerning the pathophysiology of septic shock and the associated life-threatening multiorgan failure have shown that the complement system plays a key role here—component C5a in particular is massively elevated in protracted septic shock events and is thought to be responsible inter alia for the deleterious sequelae and the progression of the disease. Recently, it has been demonstrated both experimentially and clinically that the excessive uncontrolled formation of C5a during sepsis results in consecutive paralysis of innate immunity. In the animal model, blockage of C5a by a specific antibody or receptor antagonist during sepsis increases the survival rate and leads to a milder progression of sepsis.

When the effect of taurolidine on the complement system was examined it was now found, surprisingly, that taurolidine enhances the activity of the complement system in a dose-dependent manner. In the presence of taurolidine, both the concentration and the activity of the membrane attack complex (which consists of the complement components C5b, C6, C7, C8, C9) which induces lysis of cells and microorganisms (bacteria, viruses, etc.) were increased. During this complement activation, there was a dose-dependent elevation of complement anaphylatoxin C5a (and also C3a) by taurolidine.

Thus, taurolidine is an activator of complement factor C5a or the membrane attack complex. Based on this new finding, taurolidine can no longer be recommended for use in septic shock.

However, the novel finding concerning the activation of the complement system—and therefore also the important complement component C5a—can be utilized for other therapeutic targets. Taurolidine is thus in particular a therapeutic agent for treating various forms of immunodeficiency, since the complement system is a central component of the immune defense in the human and animal organism.

The invention provides the use of taurolidine for preparing a medicament for the prevention or treatment of immunodeficiency.

The most frequent form of immunodeficiency is AIDS caused by HIV. Other innate or acquired forms of immunodeficiency are also known (see Table 1).

TABLE 1
Primary and secondary defense disorders
associated with a reduced complement system

| | |
|---|---|
| innate (primary) | complement defects: |
| | C1, C2, C4 |
| | C3, properdin |
| | C5, C6, C7, C8 |
| acquired (secondary) | infection: |
| | HIV |
| | and other viral infections |
| | immunopathies: |
| | rheumatoid arthritis |
| | systemic lupus erythematosus |
| | complement deficiency owing |
| | to autoantibodies |
| | cryoglobulinemia |
| | and other autoimmune diseases |
| | as a result of therapy: |
| | plasma separation |
| | splenectomy |
| | other disorders: |
| | nephrotic syndrome |
| | exsudative enteropathy |
| | burn trauma |

Depending on the nature and severity of the disorder, taurolidine can be administered systemically or locally. For the therapy of specific forms of immunodeficiency with qualitative or quantitative complement deficiency (for examples, see Table 1), transfusions with blood components, for example components of blood plasma, are administered, it being possible to add taurolidine to the transfusion solution, for example at an active substance content of 0.1-10% by weight to increase the effectiveness of the transferred complement components.

Taurolidine can also be used as an intravenous infusion solution, for example at an active substance content of 0.1-10% by weight. It is also possible to apply taurolidine as a solution to body cavities (abdominal cavity, pectoral cavity), to hollow viscera (bladder, intestine) or to the skin and mucous membranes. In certain cases, application may also be intrathecal.

Further forms of preparations are medicaments, for example sprays for nasal, pharyngeal and bronchial inhalation, and topical administration forms, such as cremes and gels for application to surfaces. Also possible is the administration in release systems, and also in powders and powder sprays.

The complement system also plays a role in apoptosis, the programmed cell death. This type of defense reaction against endogenous cells is of crucial importance in the suppression of excessive immune reactions and the genesis and spreading of malignant tumors. Accordingly, it is also possible to increase the activation of the complement system for suppressing excessive autoimmune disorders and for preventing the genesis and/or for preventing the spreading of cancer using taurolidine.

The invention furthermore provides the use of taurolidine for preparing a medicament for the prevention or treatment of autoimmune disorders, in particular for administration during an acute episode of the autoimmune disorder. Examples of autoimmune disorders where taurolidine may be used are rheumatoid arthritis and systemic lupus erythematosus (SLE). Pathogenetically, these two autoimmune disorders are based on the formation of immune complexes formed during complement deficiency—in particular of the classic pathway.

For treating autoimmune disorders, taurolidine is preferably administered during the acute episode as an intravenous infusion which preferably has an active substance content of 0.1-10% by weight, for example 100 ml of a 2% strength solution of taurolidine. Thereafter, the treatment can be repeated several times, for an indefinite time.

The invention furthermore provides the use of taurolidine for preparing a medicament for the prevention or treatment of malignant disorders.

For the prevention and treatment of malignant disorders, taurolidine is preferably applied as a solution having an active substance content of 0.1-10% by weight, for example as a 2% strength solution, intravenously, intrathoracally, intraabdominally or/and intraluminally (bladder, stomach, intestine). In particular, taurolidine is used as an accompanying therapeutic measure in the treatment of malignant disorders, as an adjuvans, i.e. as an accompanying therapeutic measure in combination with surgical therapy, radiation therapy or/and chemotherapy. Taurolidine can be used, for example, before, during and after cancer operations for activating phagocytosis of circulating cancer cells and dormant cancer cells—that is for preventing the recurrence of a carcinoma. This is actually a novel use of taurolidine, as hitherto only the inhibition of the spreading of metastases has been known, but not the prevention of recurrences or late recurrences.

Suitable for this treatment are in particular tumors of epithelial origin, for example carcinomas of the digestive tract, the nasal and paranasal sinuses of the nasopharyngeal space, the adenous organs, such as the mammary gland, the salivary glands, the liver including the intra- and extrahepatic bile ducts and the pancreas, the germinative tissues, such as the ovaries and testicles, the respiratory organs including the bronchial system, the lower urinary tract, such as the bladder, the ureter, the urethra, the kidneys, the uterus and certain metastasizing skin neoplasms, such as, for example, melanomas.

Even malignant neoplasms of the connective tissue, such as sarcomas of the soft tissue and the bones, the blood vessels, the nerve tissue, the neuroendocrine organs and the neuroglia cells, such as glioblastomas, and also malignant disorders of the blood or the hemopoietic cells (for example leukemias) and the lymphatic system, so-called malignant lymphomas, may be subjected to adjuvant treatment with taurolidine.

Depending on the nature and the severity of the tumor disorder, the abovementioned administration forms may be administered systemically or locally.

A further field of application of the invention is the implantation of foreign materials into a bioorganism—of human or animal. The interface between implant and live tissue is the preferred site for bacterial infections. Bacterial contamination and subsequent infection of the surface of the implant—whether of intraoperative exogen or postoperative endogen origin—regularly results in implant failure, requiring the removal of the implant. By the inventive use of taurolidine, it is possible to increase complement activity and thus phagocytosis of pathogens, which protects implants in the bioorganism against infections.

Here, taurolidine is preferably employed as a solution for rinsing having an active substance content of 0.1-10% by weight, for example as a 2% strength solution, in the implant site, but may also be applied as a powder to the implant cavity or to the surface of the implant.

A particular problem with a view to implant infections are synthetic implants having a hydrophobic surface, owing to their high affinity to problematic germs, in particular staphylococcus species. Such surfaces require a particular embodiment of the invention since taurolidine is water-soluble and usually acts in aqueous solution. By coating with endogenous proteins, it is possible to hydrophilize hydrophobic synthetic surfaces. This usually happens spontaneously when the implant is introduced into the prepared implant cavity. However, the process of hydrophilization is time-dependent, so that the biological spontaneous coating of the implant surface generally takes place in a delayed manner. In contrast, what is recommended is biological coating with endogenous proteins for hydrophilizing hydrophobic synthetic surfaces prior to implantation. This also allows the protein to be specifically selected.

This can be illustrated by an example: One of the most frequently used synthetic materials for implants having a hydrophobic surface is silicone elastomer, owing to its extraordinary stability in the bioorganism and its elastic properties. Accordingly, silicone elastomer is preferably used for producing mamma prostheses. It is known that the coagulation factors in blood—mainly fibrinogen or fibrin—have a high affinity to silicone elastomer surfaces, i.e. bind with preference to such surfaces. In this manner, they confer a hydrophilic character to the originally hydrophobic surface. To avoid implant infections, the surface of a mamma prosthesis may initially be hydrophilized as described using fibrinogen and then be introduced into the implant site with a taurolidine solution, for example a 2% strength solution. In practice, another procedure has also been successful. Here, about 1 ml of fresh blood of the patient (which contains amounts of fibrinogen sufficient for the coating procedure) is mixed with a taurolidine solution, for example with about 10 ml of 2% strength taurolidine solution. The silicone implant is incubated in this mixture for a sufficient period of time, for example about 10 min, and then—completely wetted with taurolidine solution—implanted into the organism.

In another procedure taurolidine is added as a fine powder to the silicone component of the elastomer prior to curing and finely distributed uniformly. The elastomer implant is then formed by cold or hot curing. On contact of an implant treated in this manner with blood or wound fluids taurolidine is leached from the surface of the implant while at the same time the surface is wetted with the proteins of the bodily fluids. This process is also particularly suitable for producing silicone catheters.

In this manner, it is possible to produce not only finished silicone elastomer implants but also silicone elastomer films which can be used according to the invention as coatings for implants of all types and materials (plastics, metals). This is meant to include not only permanent implants but also implants which remain in the body only for a short while, such as catheters and probes. Is is possible to coat surgical instruments which temporarily remain in the organism and thus become transient implants with appropriate films.

Finally, such taurolidine silicone elastomer films (as described) may also be used to cover wounds. A particular embodiment is the taurolidine silicone elastomer foam which is formed by foaming of the mixture during curing.

The invention also provides the use of taurolidine for preparing a medicament for preventing or treating disorders of the skin and its adnexa such as hair, especially disorders of the scalp, such as foliculitis, acne vulgaris, pyodermia, alopecia areata and also dermatomycoses.

Here, taurolidine is applied as a topical administration form, for example as a lotion, cream, gel or occluding film having an active substance concentration of preferably 0.1-10% by weight, particularly preferably about 2% by weight, to the affected regions of the skin or the hair or the scalp.

For protracted action it has been found to be expedient to add taurolidine in solid form as a powder to the gels. In this manner, a depot form is formed where taurolidine from the solid phase is dissolved and transferred into the aqueous gel medium as required.

Suitable carrier gels are all preparations customarily used in medicine and pharmacy such as, for example, gelatin, carboxymethylcellulose, polyvinyl alcohol, polyhydroxyethyl methacrylate and copolymers, alginates und polyacrylamides and mixtures of these.

### BREIF DESCRIPTION OF THE DRAWINGS

FIGS. 1-3 show a graph at hemolytic activity vs. serum dilution for the taurolidine addition of this invention;

FIGS. 4-6 show increases in complement components for such addition; and

FIG. 7 shows increase of serum concentration of MAC for such addition.

### EXAMPLE

1. Materials and Method

1.1 CH50 assay

Using the CH50 assay, it is possible to assess functionally the total activity of the complement system, especially the classic activation pathway. Here, human blood serum of healthy volunteers was incubated in a water bath at 37° C. with various concentrations of taurolidine solution (0.1-1% or the corresponding concentrations of Ringer solution for the controls) for 30 minutes. In accordance with the protocol, a dilution series (1:20-1:480) was prepared for each condition and washed sheep erythrocytes which had been preincubated with hemolysin reagent were then added. During subsequent incubation at 37° C. for exactly 1 hour the erythrocytes were lysed depending on the activity of the complement system. In each experiment, a negative control (no hemolysis), a sample with 50% hemolysis (CH50, by dilution with dist. water) and a sample with 100% hemolysis (CH100, by dilution with dist. water) were analyzed in each case. After a final centrifugation, in each case 1 ml of the supernatant of each sample was removed and the absorption at 541 nm was determined photometrically. The determined absorptions were finally plotted against the dilutions. The activity (or the ability to lyse) of the complement system under the respective conditions could be assessed by the shape of the curves (shift to the left=complement activation) and by determining the dilution at which 50% of the erythrocytes have been lysed.

1.2 ELISA

Complement anaphylatoxins C5a and C3a and the lytic complex (MAC, C5b-9) in the serum of healthy volunteers were determined quantitatively using commercially available ELISA (“enzyme-linked immunosorbent assay”) test systems. As described above, initially serum samples having various concentrations of taurolidine (0.1-5%, in parallel with samples containing Ringer solution in corresponding dilution ratios=controls) were incubated for 30 minutes at 37° C. The samples were then diluted according to the protocol of the manufacturer and pipetted into the wells of the microtiter plate. The subsequent incubation/washing steps were carried out according to the protocol of the manufacturer. Binding of the second enzyme-marked antibody resulted in a color reaction whose intensity was proportional to the C5a/C3a or MAC concentration. The extinction was measured using an ELISA plate reader and the values were then converted to concentrations using the standard samples which had also been measured.

2. Results

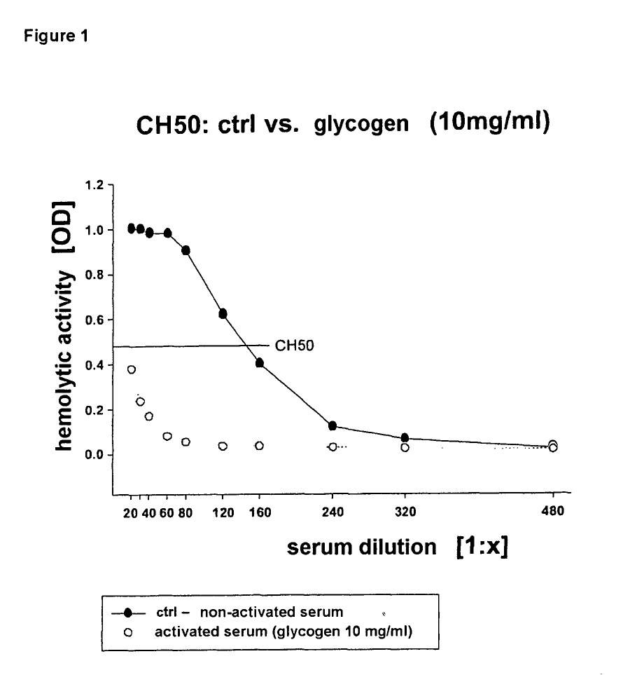

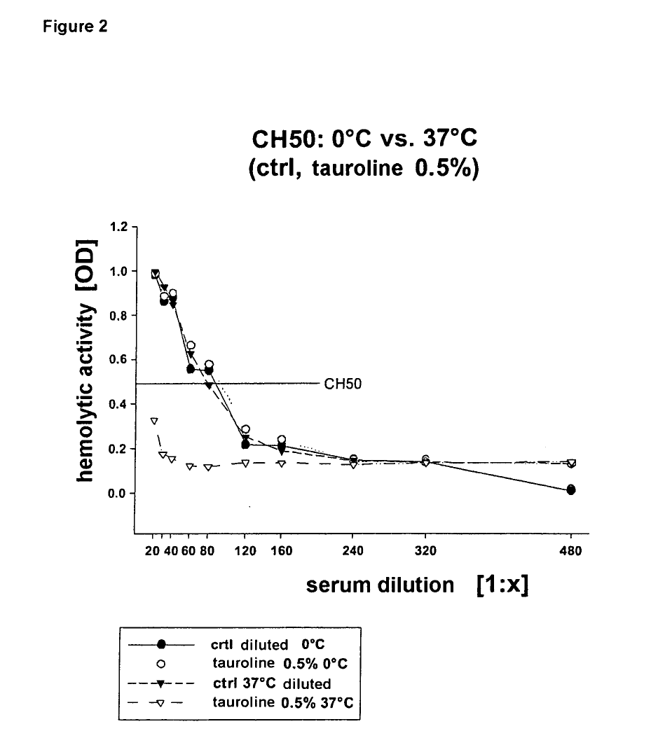

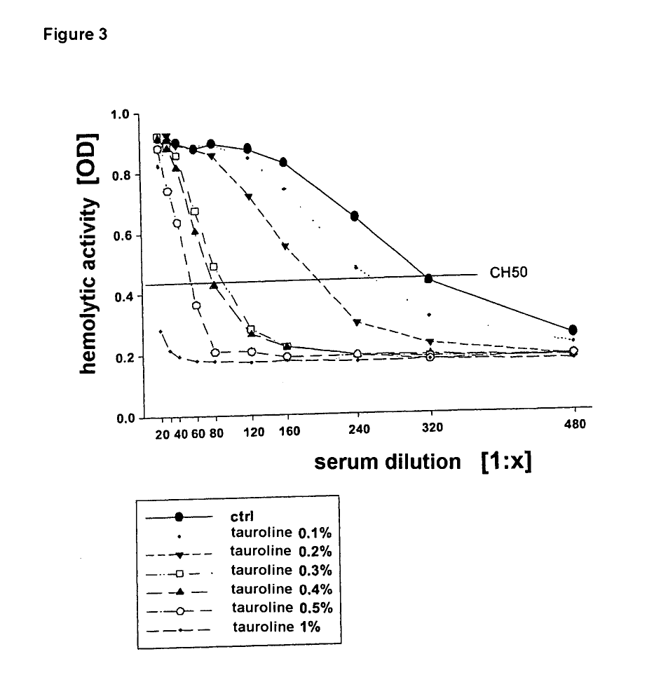

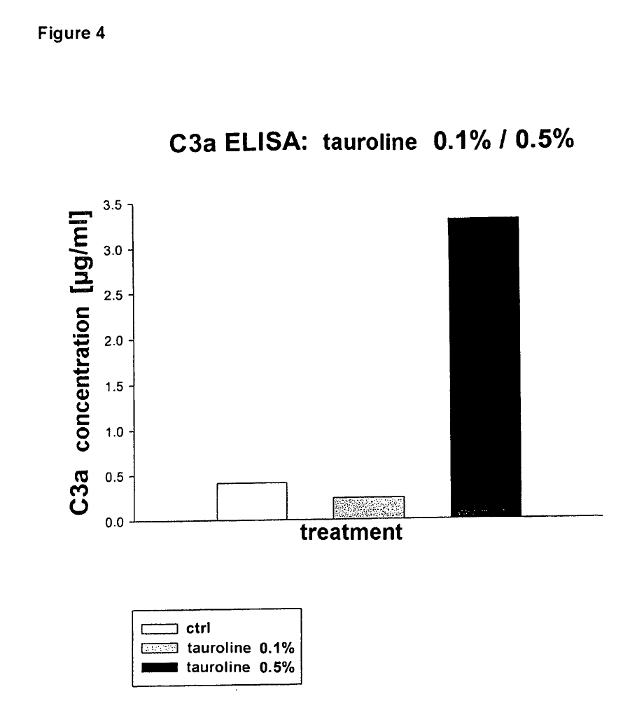

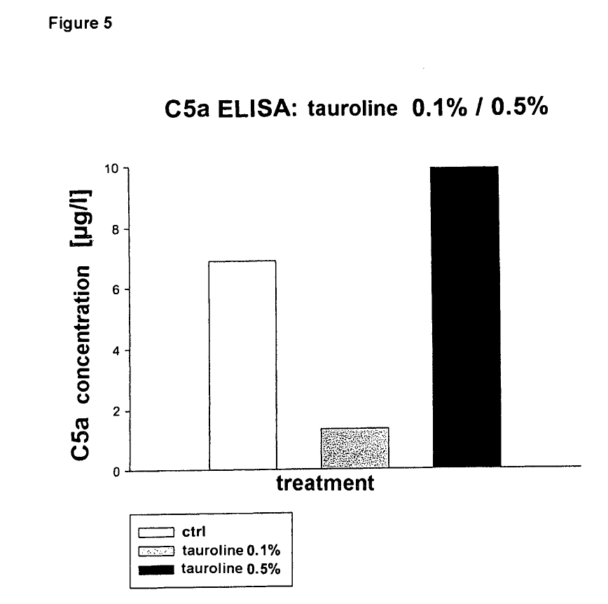

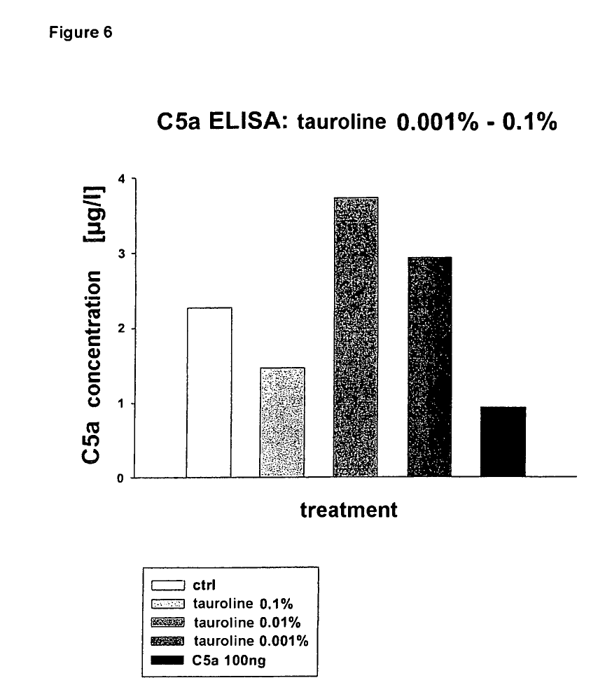

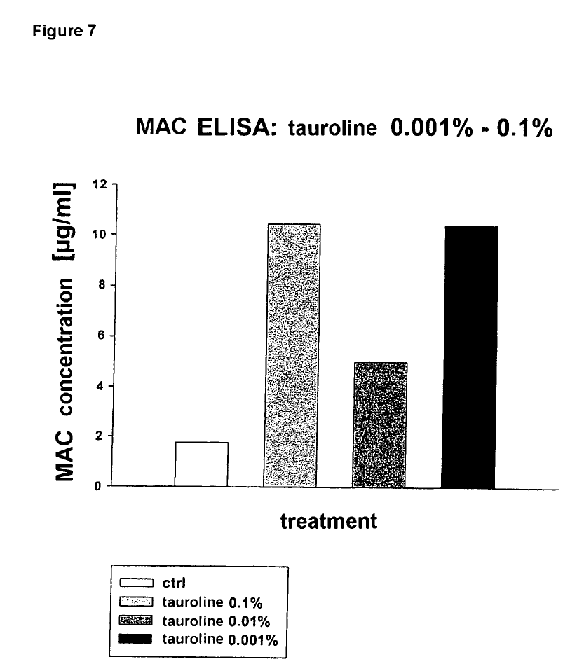

It was found that addition of taurolidine significantly increases the hemolytic activity and thus the activity of the complement system (FIGS. 1-3). This activation is temperature-dependent and takes place in particular at body temperature (37° C.), but not at 0-4° C. (FIG. 2). During complement activation by taurolidine the concentration of the complement components C3a (FIG. 4) and C5a (FIGS. 5-6) is increased compared to untreated serum (controls). Here, the increase of C3a and C5a is concentration-dependent, but not linear. Rather, there appear to be a number of concentration maxima where the presence of taurolidine enhances the formation of C3a and C5a, for example at 0.5% taurolidine. Interestingly, there is a minimum at 0.1% where no or even a tendency of reduced C3a and C5a could be observed. The reason for the different C5a concentration ranges in FIGS. 5 and 6 (in particular when compared to the controls) are interindividual differences between the subjects. In contrast to the concentrations of C3a/C5a, in all dosis ranges studied the serum concentration of the membrane attack complex (MAC) responsible for lytic activity was elevated significantly after addition of taurolidine (FIG. 7).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure of all applications, patents and publications, cited herein and U.S. Provisional Application Ser. No. 60/678,796, filed May 9, 2005, is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. The use of taurolidine for preparing a medicament for preventing or treating immunodeficiency.

2. The use as claimed in claim 1 for preventing or treating innate immunodeficiency.

3. The use as claimed in claim 1 for preventing or treating acquired immunodeficiency.

4. The use of taurolidine for preparing a medicament for preventing or treating autoimmune disorders.

5. The use as claimed in claim 4 for administration during an acute episode of an autoimmune disorder.

6. The use of taurolidine for preparing a medicament for preventing or treating malignant disorders.

7. The use as claimed in claim 6 for administration as an accompanying therapeutic measure during the treatment of malignant disorders.

8. The use as claimed in claim 6 for preventing recurrences.

9. The use of taurolidine for preparing a medicament for preventing or treating infections after implantation of foreign materials.

10. The use as claimed in claim 9, wherein an implant coated with taurolidine is used.

11. The use of taurolidine for preparing a medicament for preventing or treating disorders of the skin and its adnexa, in particular the scalp.
